(19) United States
(12) Patent Application Publication
(10) Pub. No.: US 2015/0075540 A1
(43) Pub. Date: Mar. 19, 2015

(54) APPARATUS FOR THE PREVENTION OF SLEEP APNEA

(71) Applicant: Brian Douglas Dye, Kelowna (CA)

(72) Inventor: Brian Douglas Dye, Kelowna (CA)

(21) Appl. No.: 14/029,492

(22) Filed: Sep. 17, 2013

(51) Int. Cl. A61F 5/56

(52) U.S. Cl. CPC A61F 5/566
USPC 128/848

(57) ABSTRACT

An apparatus for preventing sleep apnea in a wearer comprises upper and u-shaped appliances, a bottom protrusion extending from the lower appliance in a direction generally away from an occlusal surface of the lower appliance and a top protrusion extending from the upper appliance, the top protrusion including an adjusting block having a height of between 5/16 and 13/32 of an inch adjustably extending therefrom and having a leading surface oriented to engage the top protrusion. A method for preventing sleep apnea in a wearer comprises locating the top protrusion below and extending away from the top occlusal surface of a wearer's upper teeth, locating the bottom protrusion above and extending away from the bottom occlusal surface of a wearer's lower teeth and extending the adjusting block in an anterior direction from the top protrusion towards the bottom protrusion.

### BACKGROUND OF THE INVENTION

1. Field of Invention

The present invention relates generally to sleep apnea in general and in particular to an apparatus for preventing sleep apnea in a wearer.

2. Description of Related Art

Sleep apnea is a disorder characterized by pauses in breathing or instances of shallow or infrequent breathing during sleep. Sleep apnea disrupts the normal sleep patterns of the individual resulting in fatigue, moodiness, and decreased attention and drive. Sleep apnea can also potentially lead to serious health consequences, such as high blood pressure, heart disease and weight gain among others.

Sleep apnea commonly occurs when the soft tissue at the back of the individual's throat relaxes during sleep and collapses thereby partially or completely obstructing that person's airway. Such restriction also commonly causes the person to snore.

Conventional methods of treating sleep apnea have been to either provide supplemental oxygen to the individual while sleeping or provide a breathing device intended to reduce or prevent the restriction in their airway during sleep. Such attempts have previously been unsatisfactory. In particular, the provision of oxygen to a person typically requires providing a mask or tubes over or within the user's nostrils which may be uncomfortable for some users thereby further decreasing the amount of sleep that person is able to have in a night.

Similarly, devices intended to reduce airway restriction, have commonly focused on translating the lower jaw of the wearer in a forward direction so as to also lift the soft tissue responsible for the restriction. Such devices, however have been uncomfortable to wear by locking the patient's lower and upper jaws together thereby preventing the wearer from opening their mouth. Examples of such devices may be found in U.S. Pat. No. 7,757,693 to Toussaint and U.S. Pat. No. 5,365,945 to Halstrom.

### SUMMARY OF THE INVENTION

According to a first embodiment of the present invention there is disclosed an apparatus for preventing sleep apnea in a wearer comprising a generally u-shaped upper appliance adapted to engage with a user's upper jaw and a generally u-shaped lower appliance adapted to engage with a user's lower jaw. The apparatus further includes a bottom protrusion extending from the lower appliance in a direction generally away from an occlusal surface of the lower appliance and towards the upper appliance and a top protrusion extending from the upper appliance in a direction generally away from an occlusal surface of the upper appliance and towards the lower appliance, the top protrusion including an adjusting block having a height of between 5/16 and 13/32 of an inch adjustably extending therefrom, the adjusting block having a leading surface oriented to engage the top protrusion

The adjusting block may include an anteriorly oriented leading face. The leading face may be angled from perpendicular to the upper appliance. The leading face may be angled by 70 degrees from horizontal. The bottom protrusion may include a posteriorly oriented surface corresponding to and adapted to engage the leading face of the adjusting block.

The adjusting block may be slidably movable relative to the top protrusion. The adjusting block may include at least one slide rod extending therefrom, wherein each of the at least one slide rod is slidably supported within a corresponding bore in the top protrusion. The apparatus may further comprise two slide rods. The adjusting block may include an actuator for moving the adjusting block relative to the top protrusion. The actuator may comprise a threaded rod rotatably coupled to and extending from the adjusting block, the threaded rod being threadably received within the top protrusion.

The upper and lower appliances may be biased towards each other. The upper and lower appliances may be biased towards each other by at least one elastic band extending therebetween.

According to a first embodiment of the present invention there is disclosed a method for preventing sleep apnea in a wear comprising locating a top protrusion below and extending away from a top occlusal surface of a wearer's upper teeth, locating a bottom protrusion above and extending away from a bottom occlusal surface of a wearer's lower teeth and extending an adjusting block in an anterior direction from the top protrusion towards the bottom protrusion wherein the extension has a height of between 5/16 and 13/32 of an inch.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention wherein similar characters of reference denote corresponding parts in each view,

FIG. 1 is a perspective view of an apparatus for preventing sleep apnea in a wearer according to a first embodiment of the present invention.

FIG. 2 is a detailed exploded view of the adjusting assembly of the apparatus of FIG. 1.

FIG. 3 is a cross sectional view of the adjusting assembly of the apparatus of FIG. 1 as taken along the line 3-3.

FIG. 4 is a cross sectional view of the top and bottom teeth guards as taken along the line 4-4.

### DETAILED DESCRIPTION

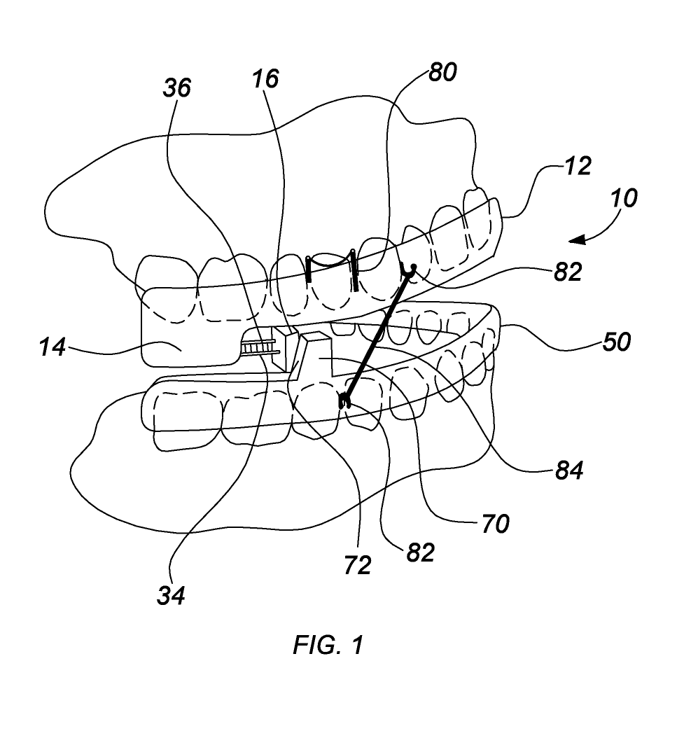

Referring to FIG. 1, an apparatus for preventing sleep apnea in a wearer according to a first embodiment of the invention is shown generally at 10. The apparatus comprises a top teeth guard 12 and a bottom teeth guard 40. The top teeth guard includes a top protrusion 14 having an adjusting block 16 extending therefrom. The adjusting block 16 is adapted engage with a corresponding bottom protrusion 70 extending from the bottom teeth guard 50.

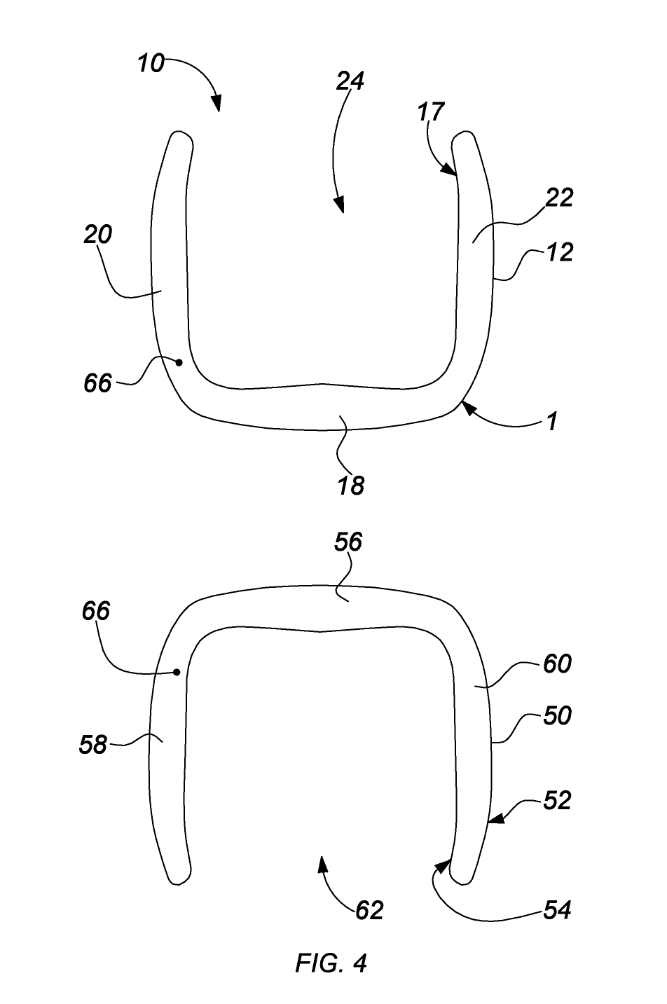

With reference to FIG. 4, the top teeth guard 12 comprises a substantially u-shaped channel member having an outer surface 15 and an inner surface 17. The top teeth guard 12 is defined by a bottom wall 18 having inner and outer walls, 20 and 22, extending therefrom. As illustrated, bottom wall 18 and inner and outer walls 20 and 22 form channel generally indicated at 24. The channel 24 is selected to substantially conform to the top teeth and/or gums of a wearer. The top teeth guard 12 may be provided as a custom or semi-custom fitted appliance for the intended wearer. In particular, the top teeth guard may be fitted to the teeth of the wearer by being heated and molded to the teeth of the wearer. Optionally, the top teeth guard 12 may be formed by first creating a mold of the wearer's top teeth and thereafter forming to such a mold. It will be appreciated that methods for forming a custom or semi-custom fitting guard as set out above are well known in the art. The top teeth guard 12 may be formed of any suitable material, such as acrylic or the like as are commonly known in the art.

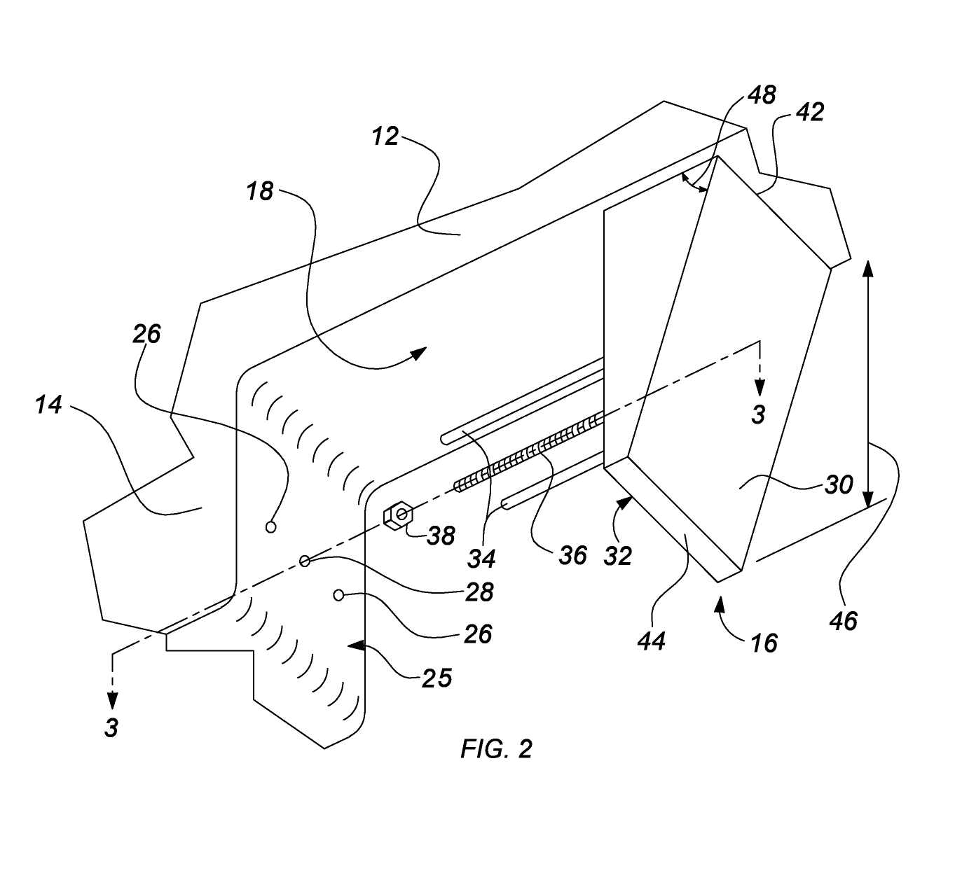

As illustrated in FIGS. 1 and 2, the top protrusion 14 comprises a block of material extending from the bottom wall 18 of the top teeth guard 12. The top protrusion 14 may be formed of the same or a different material as the remainder of the top teeth guard 12 and may be secured thereto or co-formed therewith. As illustrated in FIG. 2, the top protrusion 14 includes a front face 25 oriented towards a front of the top teeth guard 12. The top protrusion 14 includes at least one bearing bore 26 therethrough for receiving a sliding rod 34 of the adjusting block as will be more fully described below. As illustrated, the top protrusion 14 may include two bearing bores 26 therethrough although it will be appreciated that one or more than two may also be utilized. The front face also includes an adjusting bore 28 therethrough for receiving an adjusting rod 36 of the adjusting block as will be more fully described below. As illustrated in FIG. 2, the bearing bores 26 and the adjusting bore 28 may be substantially aligned with each other along a common plane although it will be appreciated that the bearing bores 26 and adjusting bore 28 may be offset from each other along as long as they are substantially parallel to each other.

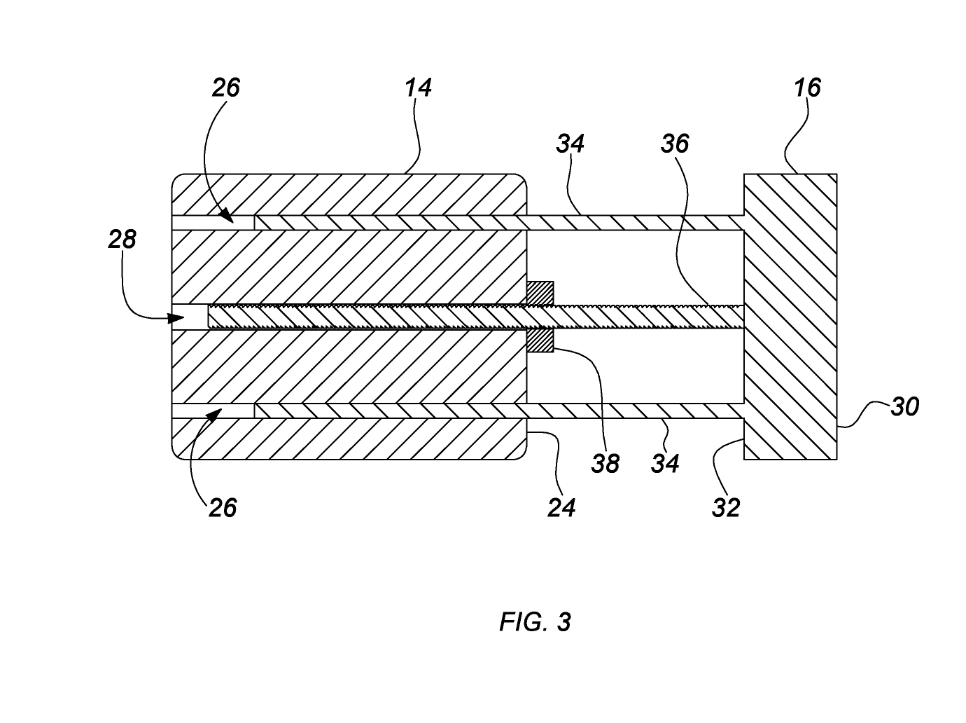

The adjusting block 16 comprises a block of material having leading and trailing faces, 30 and 32, respectively. The trailing face includes at least one sliding rod 34 extending therefrom. The adjusting block 16 may have as many sliding rods 34 as corresponding to the bearing bores 26. The sliding rods 34 have a diameter and cross-sectional profile corresponding to the bearing bores 26 so as to be slidably received therein as illustrated in FIG. 3. The bearing bores 26 may slidably support the sliding rods 34 and may optionally include bearings or bushings therein so as to facilitate such movement. The adjusting block 16 also includes and adjusting rod 36 extending from the trailing surface 32. The adjusting rod 36 may comprise a threaded rod adapted to be threadably receive within the adjusting bore 28 such that rotation of the adjusting rod 36 extends or retracts the adjusting block relative to the top protrusion 14. Optionally, the adjusting rod 36 may include a nut 38 adapted to be slidably received around the adjusting rod 36 so as to urge the adjusting rod and adjusting block 16 to a desired distance from the top protrusion 14.

The adjusting block 16 extends between top and bottom edges, 42 and 44, respectively defining the height, generally indicated at 46 of the adjusting block. The height 46 is selected to be at least 5/16 inches (8 mm) with a height of between 5/16 and 13/32 of an inch (8 and 10 mm) being particularly useful. The leading face 30 of the adjusting block 16 is also angled such adjusting block has a greater depth proximate to the top edge 42 than the bottom edge 44 by an angle below the horizontal generally indicated at 48. The angle 48 is selected to be approximately 70 degrees.

With reference to FIG. 4, the bottom teeth guard 50 comprises a substantially u-shaped channel member having an outer surface 52 and an inner surface 54. The bottom teeth guard 50 is defined by a top wall 56 having inner and outer walls, 58 and 60, extending therefrom. As illustrated, top wall 56 and inner and outer walls 58 and 60 form channel generally indicated at 62. The channel 62 is selected to substantially conform to the bottom teeth and/or gums of a wearer. Similar to the top teeth guard 12, the bottom teeth guard 50 may be provided as a custom or semi-custom fitted appliance for the intended wearer as set out above and may be made by similar materials and methods to the top teeth guard 12.

As illustrated in FIGS. 1 and 2, the bottom protrusion 70 comprises a block of material extending from the top wall 56 of the bottom teeth guard 50. The bottom protrusion 70 may be formed of the same or a different material as the remainder of the bottom teeth guard 50 and may be secured thereto or co-formed therewith. As illustrated in FIG. 1, the bottom protrusion 70 includes a rear face 72 oriented towards a rear of the bottom teeth guard 12. The bottom protrusion 70 is located at a position toward the front of the bottom and top teeth guard of the corresponding top teeth guard 12 so as to permit the rear face 72 of the bottom protrusion to be engaged upon the leading face 30 of the adjusting block 16. The rear face 72 has an angle corresponding to the angle 48 of the adjusting block such that the adjusting block and the bottom protrusion are engaged upon each other.

Optionally, the top and bottom teeth guards 12 and 50 may include one or more bracing wires 66 as illustrated in FIG. 4 and as are commonly known in the art. It will also be appreciated that the top and bottom teeth guards 12 and 50 may include clasps 80 as well as hooks 82 for securing elastic bands 84 as illustrated in FIG. 1 and as are commonly known in the art.

In operation, the top and bottom teeth guards 12 and 50 are applied to a user's top and bottom teeth as illustrated in FIG. 1. Elastic bands 84 may also be extended therebetween so as to retain the top and bottom teeth guards 12 and 50 in a proximate relation. The adjusting rod 36 is then adjusted so as to bear against the bottom protrusion and urge the bottom teeth guard 50 in an anterior direction. Such movement will thereby move the wearer's lower jaw forward thereby reducing obstruction of their airway and reducing sleep apnea.

While specific embodiments of the invention have been described and illustrated, such embodiments should be considered illustrative of the invention only and not as limiting the invention as construed in accordance with the accompanying claims.

What is claimed is:

1. An apparatus for preventing sleep apnea in a wearer comprising:
a generally u-shaped upper appliance adapted to engage with a user's upper jaw;
a generally u-shaped lower appliance adapted to engage with a user's lower jaw;
a bottom protrusion extending from said lower appliance in a direction generally away from an occlusal surface of said lower appliance and towards said upper appliance; and
a top protrusion extending from said upper appliance in a direction generally away from an occlusal surface of said upper appliance and towards said lower appliance, said top protrusion including an adjusting block having a height of between 5/16 and 13/32 of an inch adjustably extending therefrom, said adjusting block having a leading surface oriented to engage said top protrusion

2. The apparatus of claim 1 wherein said adjusting block includes an anteriorly oriented leading face.

3. The apparatus of claim 2 wherein said leading face is angled from perpendicular to said upper appliance.

4. The apparatus of claim 3 wherein said leading face is angled by 70 degrees from horizontal.

5. The apparatus of claim 3 wherein said bottom protrusion includes a posteriorly oriented surface corresponding to and adapted to engage said leading face of said adjusting block.

6. The apparatus of claim 1 wherein said adjusting block is slidably movable relative to said top protrusion.

7. The apparatus of claim 6 wherein said adjusting block includes at least one slide rod extending therefrom, wherein each of said at least one slide rod is slidably supported within a corresponding bore in said top protrusion.

8. The apparatus of claim 7 further comprising two slide rods.

9. The apparatus of claim 6 wherein said adjusting block includes an actuator for moving said adjusting block relative to said top protrusion.

10. The apparatus of claim 9 wherein said actuator comprises a threaded rod rotatably coupled to and extending from said adjusting block, said threaded rod being threadably received within said top protrusion.

11. The apparatus of claim 1 wherein said upper and lower appliances are biased towards each other.

12. The apparatus of claim 11 wherein said upper and lower appliances are biased towards each other by at least one elastic band extending therebetween.

13. A method for preventing sleep apnea in a wear comprising:
locating a top protrusion below and extending away from a top occlusal surface of a wearer's upper teeth;
locating a bottom protrusion above and extending away from a bottom occlusal surface of a wearer's lower teeth; and
extending an adjusting block in an anterior direction from said top protrusion towards said bottom protrusion wherein said extension has a height of between 5/16 and 13/32 of an inch.
